(19) United States
(12) Patent Application Publication
(10) Pub. No.: US 2019/0234921 A1
(43) Pub. Date: Aug. 1, 2019

(54) AIR QUALITY MEASURING APPARATUS

(71) Applicant: POINT ENGINEERING CO., LTD., Asan (KR)

(72) Inventors: Bum Mo AHN, Suwon (KR); Seung Ho PARK, Hwaseong (KR); Tae Hwan SONG, Cheonan (KR)

(21) Appl. No.: 16/258,453

(22) Filed: Jan. 25, 2019

(30) Foreign Application Priority Data
Jan. 31, 2018 (KR) 10-2018-0011830

(51) Int. Cl. G01N 33/00

(52) U.S. Cl. CPC G01N 33/0016; G01N 21/88; G01N 33/0031

(57) ABSTRACT

The present invention relates to an air quality measuring apparatus, which has a structure for minimizing interference between a particulate matter sensor, a gas sensor, and a humidity sensor and measures air quality by generating air flow therein by using a pumping part capable of pumping restoration.

### CROSS REFERENCE TO RELATED APPLICATION

The present application claims priority to Korean Patent Application No. 10-2018-0011830, filed Jan. 31, 2018, the entire contents of which is incorporated herein for all purposes by this reference.

### BACKGROUND OF THE INVENTION

### Field of the Invention

The present invention generally relates to an air quality measuring apparatus and, more particularly, to an air quality measuring apparatus generating air flow using a pumping part in which a sensor part and the pumping part are sequentially arranged along a direction of the air flow on an air flow passage, thereby minimizing interference in each sensor part.

### Description of the Related Art

Generally, fine dust is particulate matter (PM) floating in the atmosphere or blowing down to the ground, and the fine dust primarily occurs from burning of fossil fuels such as coal and oil or from emission gas generated from industrial plants and vehicles.

Since fine dust particulate matter (hereinafter, referred to as “particulate matter”) is invisible, the particulate matter floats in the air and penetrates lungs through respiratory organs or moves through blood vessels into the deepest parts of the human body, which can cause health problems.

In addition to the particulate matter, sick house syndrome has occurred according to increase of new buildings due to the new town developments, and interest in volatile organic compounds (VOC), which cause sick house syndrome, also has increased.

For the reasons described above, with increasing interest in air quality, an air quality measuring apparatus has been actively developed.

Various sensors for measuring air quality are provided in an air quality measuring apparatus, and different conditions and environments are required to achieve optimum performance of each of the various sensors.

As a related-art document of the present invention, Korean Patent No. 10-1006820 (registered Aug. 1, 2017, and referred to as “patent document 1” hereinbelow) is known.

The air quality measuring apparatus of patent document 1 measures air quality by generating air flow using a blower fan. In addition, an inner space of the apparatus may be used efficiently by simplifying an arrangement structure of sensors.

However, in the case of patent document 1, since the sensors do not satisfy the conditions for the optimum performance, the sensors are made of only an odor sensor and a dust sensor.

In this case, there is a limit to measurement of specific gas and humidity contained in air, and the blower fan is required to generate air flow, so that a volume of the air quality measuring apparatus is necessarily increased. Accordingly, the air quality measuring apparatus of patent document 1 has problems such as inconvenience in movement of the apparatus and reduced space efficiency.

The foregoing is intended merely to aid in the understanding of the background of the present invention, and is not intended to mean that the present invention falls within the purview of the related art that is already known to those skilled in the art.

### DOCUMENTS OF RELATED ART

(Patent Document 1) Korean Patent No. 10-1006820 (Dec. 31, 2010).

### SUMMARY OF THE INVENTION

Accordingly, the present invention has been made keeping in mind the above problems occurring in the related art, and the present invention is intended to propose an air quality measuring apparatus that is able to provide an arrangement structure for minimizing interference of each sensor and to achieve minimization in the size of the air quality measuring apparatus.

In order to achieve the above object, according to one aspect of the present invention, there is provided an air quality measuring apparatus that includes: an air flow passage provided with an air inlet and an air outlet respectively formed at both ends thereof; a pumping part provided to bring outside air into the air flow passage through the air inlet and to discharge inside air through the air outlet; and a sensor provided between the pumping part and the air inlet, and measuring particles in outside air, wherein the air inlet, the sensor, the pumping part, and the air outlet may be sequentially arranged on the air flow passage.

The sensor may be at least one of a particulate matter sensor, a gas sensor, and a humidity sensor.

The sensor may be a particulate matter sensor and a gas sensor.

The particulate matter sensor and the gas sensor may be arranged in series on the air flow passage.

The particulate matter sensor and the gas sensor may be arranged in parallel on the air flow passage.

The sensor may be a particulate matter sensor or a gas sensor, and a moisture filter may be arranged between the air inlet and the gas and particulate matter sensors.

The pumping part may include a first pumping part and a second pumping part, and the sensor may include a particulate matter sensor, a gas sensor, and a humidity sensor, wherein the first pumping part may be connected to the particulate matter sensor and the humidity sensor, and the second pumping part may be connected to the gas sensor.

As described above, the air quality measuring apparatus according to the embodiment of the present invention has following effects.

Since the sensor parts are arranged in series on the air flow passage, interference between the sensor parts can be minimized, and accurate measurement of each sensor part can be expected. In addition, the sensor parts arranged in a line on the air flow passage realize a simple arrangement, thereby being easy to manufacture.

In addition, since the sensor parts are arranged in parallel, the first sensor part and the second sensor part simultaneously receive and measure outside air, that is, air that is subject to measurement, thereby reducing measurement time. As the length of the air flow passage is reduced, the required capacity of the pumping part may be reduced, which is advantageous for minimization in the size of the apparatus.

In addition, an error occurring by moisture particles can be reduced to a minimum by providing the humidity sensor, the gas sensor, and the particulate matter sensor in separated spaces.

In addition, as a separate pumping part is provided and an amount difference is made in the pumping part to generate air flow, the same amount of air is supplied to the first and second air flow passages, thereby reducing an error in measured values.

### BRIEF DESCRIPTION OF THE DRAWINGS

The above and other objects, features and other advantages of the present invention will be more clearly understood from the following detailed description when taken in conjunction with the accompanying drawings, in which:

FIG. 1A is a vertical cross-sectional view showing an air quality measuring apparatus according to a first exemplary embodiment of the present invention.

FIG. 1B is a schematic view showing a structure of the apparatus in FIG. 1A.

FIG. 2A is a horizontal cross-sectional view showing an air quality measuring apparatus according to a second exemplary embodiment of the present invention.

FIG. 2B is a schematic view showing a structure of the apparatus in FIG. 2A.

FIG. 3A is a horizontal cross-sectional view showing an air quality measuring apparatus according to a third exemplary embodiment of the present invention.

FIG. 3B is a schematic view showing a structure of the apparatus in FIG. 3A.

### DETAILED DESCRIPTION OF THE INVENTION

Hereinbelow, exemplary embodiments of the present invention will be described in detail with reference to the accompanying drawings.

In the following description, only the theory of the present invention will be described. Thus, although some embodiments are not clearly described in the following description or not clearly shown in the accompanying drawings, those skilled in the art can provide various apparatuses that may be configured to embody the theory of the present invention within the scope and spirit thereof. Further, the terminology used herein is for the purpose of describing particular aspects or embodiments of the present invention only and is not intended to be limiting of the present invention.

The above and other objects, features and other advantages of the present invention will be more clearly understood from the following detailed description when taken in conjunction with the accompanying drawings, and those skilled in the art will easily understand and may embody the scope and spirit of the invention.

An Air Quality Measuring Apparatus 100 According to a First Exemplary Embodiment of the Present Invention

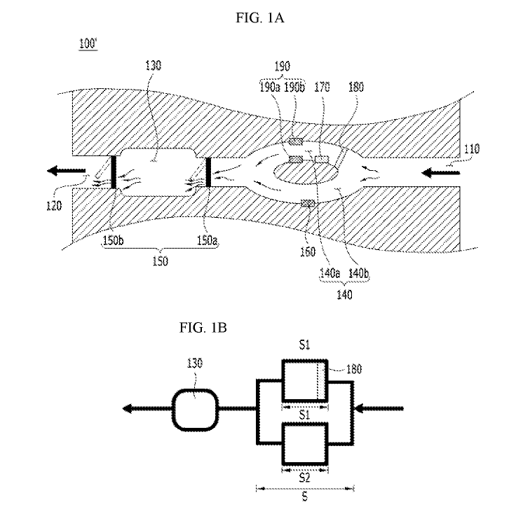

First, an air quality measuring apparatus 100 according to a first exemplary embodiment of the present invention will be described with reference to FIG. 1.

FIG. 1A is a vertical cross-sectional view showing the air quality measuring apparatus according to the first exemplary embodiment of the present invention. FIG. 1B is a schematic view showing a structure of the apparatus in FIG. 1A.

The air quality measuring apparatus 100 according to the first exemplary embodiment of the present invention includes an air flow passage 140 provided with an air inlet 110 and an air outlet 120 respectively formed at opposite ends thereof, a pumping part 130 provided to bring outside air into the air flow passage 140 through the air inlet 110 and to discharge inside air through the air outlet 120, and a sensor provided between the pumping part 130 and the air inlet 110, and measuring particles contained in the outside air.

The air inlet 110 communicates with a sensor part S, and functions as a passage through which outside air flows into the air quality measuring apparatus 100.

The air outlet 120 communicates with the pumping part 130 and functions as a passage that discharges inside air of the air quality measuring apparatus 100.

The air flow passage 140 functions as a passage through which the air inlet 110 and the air outlet 120 communicate with each other.

The air flow passage 140 has the air inlet 110 and the air outlet 120 at the opposite ends thereof.

The air flow passage 140 has the air inlet 110 at a first end thereof and the air outlet 120 at a second end. The air flow passage 140 formed between the air inlet 110 and the air outlet 120 includes the sensor part S measuring air quality and the pumping part 130 generating air flow.

The sensor part S includes a first sensor part S1 and a second sensor part S2.

The first sensor part S1 may include a humidity sensor 160 measuring humidity, and the second sensor part S2 may include a particulate matter sensor 190 measuring particulate matter and a gas sensor 170 measuring gas.

The humidity sensor 160 functions to measure humidity in the air.

It is preferable that the humidity sensor 160 is a capacitance-type humidity sensor 160.

This is because the capacitance-type humidity sensor 160 is capable of sensing in a low humidity condition, does not need temperature control, and has a thin film structure thereby responding quickly. In addition, since the capacitance-type humidity sensor 160 may sense humidity of air quality in real time, it is easy to use in a portable air quality measuring apparatus 100.

The gas sensor 170 includes a sensing film reacting to a specific gas and a heater generating heat.

The sensing film reacts to the specific gas at a predetermined temperature. Therefore, by providing the gas sensor 170 and the heater together, it is possible to measure a specific gas in the air through temperature control.

A moisture filter 180 is arranged between the air inlet 110 and the gas and particulate matter sensors 170 and 190.

The moisture filter 180 functions to filter moisture contained in outside air flowing through the air inlet 110.

The particulate matter sensor 190 measures particulate matter contained in the air using a light scattering method capable of real time measurement.

The particulate matter sensor 190 is provided with a light emitter 190a and a light receiver 190b, as shown in FIG. 1A, wherein the light emitter 190a and the light receiver 190b are provided on opposite side surfaces of the air flow passage 140.

For example, when the light emitter 190a is provided on a first surface of the air flow passage 140, the light receiver 190b is provided on a second surface thereof.

The light emitter 190a emits light, and the light receiver 190b collects the emitted light from the light emitter 190a.

It is preferable that the light emitter 190a and the light receiver 190b are arranged in parallel to face each other.

When the outside air flowing into the particulate matter sensor 190 passes through the light emitter 190a and the light receiver 190b, light emitted from the light emitter 190a is scattered by particulate matter in the outside air.

As the light is scattered by the particulate matter, the amount of light reaching the light receiver 190b varies, therefore concentration of the particulate matter is measured by detecting the amount of the light reaching the light receiver 190b.

In other words, when the concentration of the particulate matter in the air is high, the light reaching the light receiver 190b is detected in a small amount, and when the concentration of the particulate matter in the air is low, the light reaching the light receiver 190b is detected in a large amount, and the result is used to measure concentration of the particulate matter.

In a measurement method of the concentration of the particulate matter as described above, error may occur because the light is also scattered by the moisture particles in the air.

Consequently, since the moisture particles in the air may be removed in advance by using heat of the gas sensor 170, the error of the particulate matter sensor 190 can be greatly reduced.

Therefore, it is preferable that the sensors are provided in the order of the gas sensor 170 and the particulate matter sensor 190, and particles contained in the air may be measured more accurately.

The pumping part 13 is a device for generating air flow in the air quality measuring apparatus 100.

The pumping part 13 may be made of an elastic member having an elastic force, and the air quality measuring apparatus 100 may generate air flow therein by using elastic restoring force of the elastic member.

The pumping part 13 of an elastic material may be formed of a material such as rubber, but it is not limited thereto.

A check valve 150 includes a first check valve 150a and a second check valve 150b.

The first check valve 150a is arranged between the sensor part S and the pumping part 130, and the second check valve 150b is arranged between the pumping part 130 and the air outlet 120.

The first check valve 150a divides the pumping part 130 and the sensor part S as a separate space and performs as a reference point for dividing the pumping part 130 and the sensor part S on the air flow passage 140.

Hereinafter, arrangement order of the component described above will be described.

In case of the air quality measuring apparatus 100 according to the first exemplary embodiment of the present invention, the air inlet 110, the first sensor part S1, the second sensor part S2, the first check valve 150a, the pumping part 130, the second check valve 150a, and the air outlet 120 are sequentially arranged as shown in FIG. 1.

The sensor part S and the pumping part 130 are arranged on the air flow passage 140 in the order of the sensor part S and the pumping part 130.

In the air quality measuring apparatus 100, air flow proceeds in a way that residual air in the pumping part 130 is discharged and then outside air flows through the air inlet 110.

When the pumping part 130 is arranged before the sensor part S on the air flow passage 140, the sensors provided in the sensor part S measures air remaining in the pumping part 130, that is, air remaining in the air quality measuring apparatus 100 first.

Accordingly, with arranging the pumping part 130 after the sensor part S on the air flow passage 140, air quality may be preferably measured by discharging residual air in the air quality measuring apparatus 100 through the air outlet 120 and then bringing new outside air through the air inlet 110.

The first check valve 150a is only opened in the direction of the pumping part 130, prevents a back flow of the air remaining in the pumping part 130 while providing between the sensor S and the pumping part 130, and divides into the pumping part 130 and the sensor S as separate spaces. Such a first check valve 150a may be operated by suction force of the pumping part 130. However, the first check valve 150a is not limited thereto, but may be operated by another operating force capable of generating one-way air flow.

The second check valve 150b is only opened in the direction of the air outlet 120 by compression force acting on the pumping part 130, and arranged between the pumping part 130 and the air outlet 120 to prevent outside air from flowing into the air outlet 120.

Such second check valve 150b may be operated by the compression force of the pumping part 130. However, the second check valve 150b is not limited thereto, but may be operated by another operating force capable of generating one-way air flow.

Specifically, air flow is generated in the air quality measuring apparatus 100 by operation of the pumping part 130. However, when the first check valve 150a is not provided, inside air remaining in the pumping part 130 flows backward in the direction of the air outlet 110 by compression.

In this case, an error may occur such that the inside air of the pumping part 130 is measured first in the sensor part S.

In addition, when the second check valve 150b is not provided while the suction force is generated by the pumping part, outside air flows through the air outlet 120.

Accordingly, by providing the check valve 150 between the sensor part S and the pumping part 130 and the air outlet 120, back flow of air is prevented in the air quality measuring apparatus 100 and one-way air flow may be induced.

The sensor part S is arranged in the order of the first sensor part S1 and the second sensor part S2.

The sensor part S is arranged in an air flow direction in the order of the humidity sensor 160, the gas sensor 170, and the particulate matter sensor 190, and arranged in a line on the air flow passage 140.

The humidity sensor 160 is arranged after the air inlet 110 in the air flow direction.

The humidity sensor 160 is affected by the moisture filter 180, therefore, it is preferable that the humidity sensor 160 is arranged before the moisture filter 180, and before the gas sensor 170.

When the gas sensor 170 is arranged before the humidity sensor 160, it is difficult to measure accurate humidity due to heat generated by the heater of the gas sensor 170. Thus, it is preferred that the sensor part S is arranged in the order of the humidity sensor 160 and the gas sensor 170.

The gas sensor 170 is arranged after the moisture filter 180.

Specifically, sensitivity of the sensing film of the gas sensor 170 may be decreased due to moisture particles contained in the air thereby causing an error in measured values. However, the error of the gas sensor 170 may be reduced by providing the moisture filter 180 between the first sensor S1 and the second sensor S2, that is, between the humidity sensor 160 and the gas sensor 170.

The particulate matter sensor 190 is affected by moisture particles. Therefore, as described above, since the moisture particles are removed by the heat generated in the gas sensor 170, it is preferable that the particulate matter sensor 190 is arranged after the gas sensor 170.

That is, on the air flow passage 140, the sensor part S is arranged in the order of the humidity sensor 160, the moisture filter 180, the gas sensor 170, and particulate matter sensor 190 in series.

As described above, in the air quality measuring apparatus 100 according to the first embodiment of the present invention, interference between each sensor part S may be minimized and accurate measurement may be expected by arranging the sensor part S in series on the air flow passage 140. In addition, the sensor part S arranged in a line on the air flow passage 140 is simple in arrangement and easy to manufacture.

An Air Quality Measuring Apparatus 100′ According to a Second Exemplary Embodiment of the Present Invention

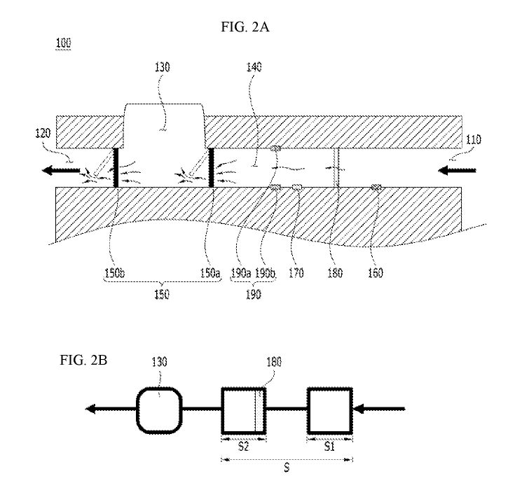

Hereinafter, an air quality measuring apparatus 100′ according to a second exemplary embodiment of the present invention will be described with reference to FIG. 2.

FIG. 2A is a horizontal cross-sectional view showing the air quality measuring apparatus 100′ according to the second exemplary embodiment of the present invention. FIG. 2B is a schematic view showing a structure of the apparatus in FIG. 2A.

As shown in FIG. 2, the air quality measuring apparatus 100′ is different from the above-mentioned air quality measuring apparatus 100 in aspects of a shape of an air flow passage 140 and an arrangement structure of a sensor part S, but it is the same in aspects of the rest of components and effects.

Thus, the same components may be replaced by the foregoing description, and redundant descriptions are omitted.

As shown in FIG. 2, in the air quality measuring apparatus 100′ according to the second exemplary embodiment of the present invention, a humidity sensor 160 is arranged with a particulate matter sensor 190, and a gas sensor 170 in parallel on an air flow passage 140.

A sensor part S of the air quality measuring apparatus 100′ is arranged in parallel.

The air flow passage 140 is divided into a first air flow passage 140a and a second air flow passage 140b, and two passages join into one air flow passage 140 after passing through the sensor part S, and then connected to a pumping part 130.

The sensor part S is provided with a first sensor part S1 and a second sensor part S2.

For example, the first sensor part S1 is gas sensor 170 and the particulate matter sensor 190, and the second sensor part S2 is the humidity sensor 160.

The first air flow passage 140a has the gas sensor 170 and the particulate matter sensor 190, and second air flow passage 140b has the humidity sensor 160. In addition, a moisture filter 180 is provided at the entrance of the first air flow passage 140a so that it filters moisture particles flowing into the first air flow passage 140a.

With arranging each sensor part S in parallel, outside air, that is, air that is subject to measure flows and is measured by the first sensor part S1 and the second sensor part S2 at the same time, thus, measurement time may be reduced. In addition, a length of the air flow passage 140 is reduced, thus, the required capacity of the pumping part 130 may be provided to be relatively smaller than the air quality measuring apparatus 100 arranged in series, which is advantageous for minimization in the size of the apparatus.

In addition, with arranging the gas sensor 170 and the particulate matter sensor 190 in a separate space from the humidity sensor 160, error that occurs due to moisture particles from the gas sensor 170 and the particulate matter sensor 190 may be reduced to a minimum.

An Air Quality Measuring Apparatus 100″ According to a Third Exemplary Embodiment of the Present Invention

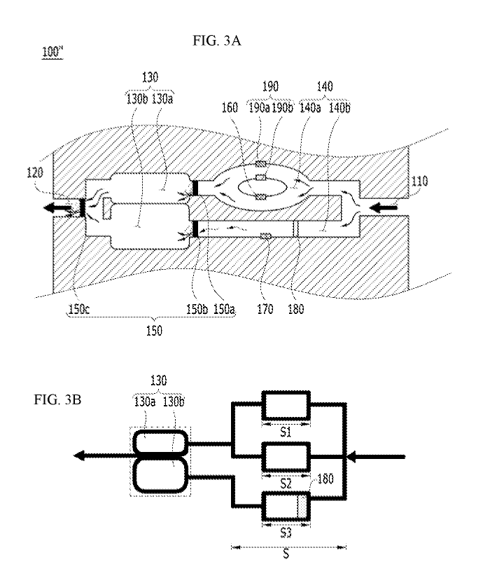

Hereinafter, an air quality measuring apparatus 100″ according to a third exemplary embodiment of the present invention will be described with reference to FIG. 3.

FIG. 3A is a horizontal cross-sectional view showing the air quality measuring apparatus according to the third exemplary embodiment of the present invention. FIG. 3B is a schematic view showing a structure of the apparatus in FIG. 3A.

As shown in FIG. 3, the air quality measuring apparatus 100″ is different from other embodiments in aspects of a shape of a pumping part 130, a shape of an air flow passage 140, and an arrangement structure of a sensor part S, but it is the same in aspects of the rest of components and effects.

Thus, the same component may be replaced by the foregoing description, and redundant descriptions are omitted.

In the air quality measuring apparatus 100″ according to the third exemplary embodiment of the present invention, the pumping part 130 includes a first pumping part 130a and a second pumping part 130b, and a sensor part S includes a first sensor part S1, a second sensor part S2, and a third sensor part S3.

The pumping part 130 includes the first pumping part 130a communicating with a first air flow passage 140a and a second pumping part 130b communicating with a second air flow passage 140b.

The pumping part 130 is divided into the first pumping part 130a and second pumping part 130b, but the first and second pumping parts 130a and 130b may be simultaneously operated when the pumping part 130 is operated.

That is, an inner space of one pumping part 130 is divided into the first pumping part 130a and the second pumping part 130b.

Capacity of pumping part 130 is formed such that capacity of second pumping part 130b is relatively larger than capacity of first pumping part 130a.

The air flow passage includes the first air flow passage 140a communicating with the first pumping part 130a and the second air flow passage 140b communicating with the second pumping part 130b.

The first air flow passage 140a has the first sensor part S1 and the second sensor part S2, and the second air flow passage 140b has the third sensor part S3.

The first sensor part S1 and the second sensor part S2 are respectively provided on an air flow passage 140 that is divided into two passages from the first air flow passage 140a and joins to one first air flow passage 140a again after passing through the sensor part, thus the first sensor part S1 and the second sensor part S2 have a separate space, respectively.

For example, the first sensor part S1 may be a particulate matter sensor 190, the second sensor part S2 may be a humidity sensor 160, and the third sensor part S3 may be a gas sensor 170.

A check valve 150 includes a first check valve 150a provided on the first air flow passage 140a, a second check valve 150b provided on the second air flow passage 140b, and a third check valve 150c arranged between the pumping part 130 and an air outlet 120.

The first check valve 150a is provided between the first pumping part 130a and the first and second sensor parts S1 and S2, and separates the first pumping part 130a and the first and second sensor parts S1 and S2.

The second check valve 150b is provided between the second pumping part 130b and the third sensor part S3, and separates the second pumping part 130b and the third sensor part S3.

The third check valve 150c is provided between the pumping part 130 and the air outlet 120, and prevents outside air from flowing through the air outlet 120.

In addition, the first and second check valves 150a and 150b are opened only in a direction of the pumping part 130 by suction force of the pumping part 130, and the third check valve 150c is opened in a direction of the air outlet 120 by compression force of the pumping part 130. Therefore, one-way air flow may be generated in the air quality measuring apparatus 100″.

As described above, since the air quality measuring apparatus 100″ according to the third embodiment of the present invention is provided such that the sensor part S having a parallel structure is separately connected to the pumping part 130, the air quality measuring apparatus 100″ may supply air to the first air flow passage 140a and the second air flow passage 140b, respectively.

In the air quality measuring apparatus 100″ having a parallel structure in one pumping part 130, air resistance is generated by a moisture filter 180, so a difference may occur in amounts of air supplied to the first air flow passage 140a and the second air flow passage 140b. That is, when air resistance force is generated due to the moisture filter 180 on the second air flow passage 140b, more outside air may flow in the first air flow passage 140a.

In this case, a sensor provided on the second air flow passage 140b may not receive enough air to measure air quality.

Accordingly, as the separate pumping parts 130 are provided at the first air flow passage 140a and the second air flow passage 140b and capacity of the second pumping part 130b is provided to be relatively large, compression, that is, suction force larger than the first air flow passage 140a is generated in the second air flow passage 140b, and the same amount of air is supplied to the first air flow passage 140a and the second air flow passage 140b, thereby reducing errors in measurement valves.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. An air quality measuring apparatus comprising:
an air flow passage provided with an air inlet and an air outlet respectively formed at both ends thereof;
a pumping part provided to bring outside air into the air flow passage through the air inlet and to discharge inside air through the air outlet; and
a sensor provided between the pumping part and the air inlet, and measuring particles included in the outside air,
wherein the air inlet, the sensor, the pumping part, and the air outlet are sequentially arranged on the air flow passage.

2. The air quality measuring apparatus of claim 1, wherein the sensor is at least one of a particulate matter sensor, a gas sensor, and a humidity sensor.

3. The air quality measuring apparatus of claim 1, wherein the sensor comprises a particulate matter sensor and a gas sensor.

4. The air quality measuring apparatus of claim 3, wherein the particulate matter sensor and the gas sensor are arranged in series on the air flow passage.

5. The air quality measuring apparatus of claim 3, wherein the particulate matter sensor and the gas sensor are arranged in parallel on the air flow passage.

6. The air quality measuring apparatus of claim 1, wherein the sensor is a particulate matter sensor or a gas sensor, and a moisture filter is arranged between the air inlet and the gas and particulate matter sensors.

7. The air quality measuring apparatus of claim 1, wherein the pumping part comprises a first pumping part and a second pumping part, and the sensor comprises a particulate matter sensor, a gas sensor, and a humidity sensor, wherein the first pumping part is connected to the particulate matter sensor and the humidity sensor, and the second pumping part is connected to the gas sensor.
